(19) United States
(12) Patent Application Publication
(10) Pub. No.: US 2022/0307068 A1
(43) Pub. Date: Sep. 29, 2022

(54) GENE AMPLIFICATION CHIP, APPARATUS FOR GENE AMPLIFICATION, AND METHOD OF MANUFACTURING GENE AMPLIFICATION CHIP

(71) Applicant: SAMSUNG ELECTRONICS CO., LTD., Suwon-si (KR)

(72) Inventors: Won Jong JUNG, Seoul (KR); Kak NAMKOONG, Seoul (KR); Young Zoon YOON, Hwaseong-si (KR)

(73) Assignee: SAMSUNG ELECTRONICS CO., LTD., Suwon-si (KR)

(21) Appl. No.: 17/356,126

(22) Filed: Jun. 23, 2021

(30) Foreign Application Priority Data
Mar. 24, 2021 (KR) 10-2021-0037923

(51) Int. Cl. C12Q 1/6806; B01L 7/00; G01N 21/64; B01L 3/00; G01N 1/28

(52) U.S. Cl. CPC C12Q 1/6806; B01L 7/52; G01N 21/6452; B01L 3/502715; G01N 1/28; B01L 2300/0829; B01L 2300/0851; B01L 2300/1822; B82Y 5/00

(57) ABSTRACT

A gene amplification chip may include a substrate; a through-hole array including through-holes that extend from an upper surface of the substrate to a lower surface of the substrate and in which a gene amplification reaction occurs; and a photothermal film provided on at least one of the upper surface and the lower surface of the substrate and configured to generate heat using light.

### CROSS-REFERENCE TO RELATED APPLICATION(S)

This application claims priority to Korean Patent Application No. 10-2021-0037923, filed on Mar. 24, 2021, in the Korean Intellectual Property Office, the entire disclosure of which is incorporated by reference herein for all purposes.

### BACKGROUND

### 1. Field

Example embodiments of the present disclosure relate to a gene amplification chip and apparatus.

### 2. Description of Related Art

Sample analysis for medical or environmental purposes is executed through a series of biochemical, chemical, and mechanical processes. Recently, technologies for diagnosing or monitoring biological samples have been actively developed. Due to high accuracy and sensitivity requirements, a molecular diagnosis method based on a nucleic acid is increasingly and broadly being used to diagnose infectious diseases and cancers to study pharmacogenomics, as well as to develop new medicines. Microfluidic devices are widely used to analyze a sample in a simple and precise manner.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to an aspect of an example embodiment, a gene amplification chip may include a substrate; a through-hole array including through-holes that extend from an upper surface of the substrate to a lower surface of the substrate and in which a gene amplification reaction occurs; and a photothermal film provided on at least one of the upper surface and the lower surface of the substrate and configured to generate heat using light.

The substrate may comprise silicon (Si), glass, polymer, or metal.

A thickness of the substrate may be less than or equal to 1 millimeter (mm).

A respective volume of each through-hole may be less than or equal to 1 nanoliter (nL).

A number of through-holes may be equal to or greater than 20,000.

The through-holes may be provided in the shape of a circular cylinder or a polygonal cylinder.

The through-holes may be provided in the shape of a hexagonal cylinder, a diagonal distance of a cross-sectional area of each through-hole is less than or equal to 100 micrometers (μm).

A thickness of the photothermal film may be less than or equal to 10 micrometers (μm).

The photothermal film may be provided on partition walls of each of the through-holes.

The photothermal film may comprise a metal layer.

The photothermal film may comprise nanoparticles, nanorods, nanodisks, or nanoislands.

The gene amplification chip may further comprise an auxiliary film attached to the photothermal film.

The auxiliary film may be comprised of silicon dioxide (SiO₂), titanium dioxide (TiO₂), tantalum dioxide (TaO₂), silicon nitride (SiN), or polymer.

The gene amplification chip may further comprise an adhesive film disposed between the substrate and the photothermal film to provide adhesion of the photothermal film.

According to an aspect of another example embodiment, an apparatus for gene amplification may include a main body; a gene amplification chip; a chamber provided on a side of the main body, formed to allow the gene amplification chip to be inserted therein, and connected to a solution inlet and a solution outlet through fluid conduits; a light source configured to emit light to the gene amplification chip; and a detector configured to detect fluorescence emitted from an amplified gene. The gene amplification chip may comprise a substrate; a through-hole array including through-holes that extend from an upper surface of the substrate to a lower surface of the substrate and in which a gene amplification reaction occurs; and a photothermal film provided on at least one of the upper surface and the lower surface of the substrate and configured to generate heat using light.

The chamber may comprise an upper surface and a lower surface, and the gene amplification chip is inserted between the upper surface and the lower surface.

When a solution is loaded through the solution inlet and introduced into the chamber along the fluid conduits, the solution may be injected into the through-holes by capillary action.

The apparatus may further comprise a cutter configured to discharge solution remaining in the chamber other than in the inside of the through-holes to the solution outlet after the solution loaded through the solution inlet is injected into the through-holes.

The apparatus may further comprise a light source controller configured to heat and cool the photothermal film by driving the light source in an on-off manner.

The photothermal film may reflect the fluorescence emitted from the gene amplified inside the through-holes in a direction of the detector.

According to an aspect of another example embodiment, a method of manufacturing a gene amplification chip may include forming through-holes in a substrate, the through-holes extending in a direction from an upper surface to a lower surface of the substrate; planarizing the lower surface of the substrate using a chemical mechanical polishing (CMP) process; and depositing a photothermal film on at least one of the upper surface and the lower surface of the substrate.

The method may further comprise depositing the photothermal film on partition walls of each of the through-holes.

Other features and aspects will be apparent from the following detailed description, the drawings, and the claims.

### BRIEF DESCRIPTION OF THE DRAWINGS

The above and other aspects, features, and advantages of certain example embodiments of the present disclosure will be more apparent from the following description taken in conjunction with the accompanying drawings, in which:

FIG. 1 is a diagram illustrating a gene amplification chip according to an example embodiment;

FIG. 2 is a side view of a gene amplification chip with a photothermal film deposited thereon;

FIG. 3 is a diagram illustrating a gene amplification chip according to another example embodiment;

FIG. 4 is a diagram illustrating an apparatus for gene amplification according to an example embodiment;

FIG. 5 is a side view of a chamber shown in FIG. 4;

FIGS. 6A to 6E illustrate a process in which a solution is injected into through-holes;

FIGS. 6F to 6J illustrate a process in which a solution remaining in a chamber other than the inside of through-holes is discharged by a cutter to a solution outlet;

FIG. 7 is a block diagram illustrating an apparatus for gene amplification according to an example embodiment;

FIG. 8 is a block diagram illustrating an apparatus for gene amplification according to another example embodiment; and

FIG. 9 is a flowchart illustrating a method of manufacturing a gene amplification chip according to an example embodiment.

### DETAILED DESCRIPTION

Details of example embodiments are provided in the following detailed description with reference to the accompanying drawings. The disclosure may be understood more readily by reference to the following detailed description of example embodiments and the accompanying drawings. The disclosure may, however, be embodied in many different forms and should not be construed as being limited to the example embodiments set forth herein. Rather, these example embodiments are provided so that the disclosure will be thorough and complete and will fully convey the concept of the present disclosure to those skilled in the art, and the present disclosure will only be defined by the appended claims.

Throughout the drawings and the detailed description, unless otherwise described, the same drawing reference numerals will be understood to refer to the same elements, features, and structures. The relative size and depiction of these elements, features, and structures may be exaggerated for clarity, illustration, and convenience.

It will be understood that, although the terms “first,” “second,” etc., may be used herein to describe various elements, these elements should not be limited by these terms. These terms are only used to distinguish one element from another. Also, the singular forms of terms are intended to include the plural forms of terms as well, unless the context clearly indicates otherwise. In the specification, unless explicitly described to the contrary, the word “comprise,” “include”, and variations such as “comprises,” “comprising,” “includes,” or “including,” will be understood to imply the inclusion of stated elements but not the exclusion of any other elements. Terms such as “unit” and “module” denote units that process at least one function or operation, and they may be implemented by using hardware, software, or a combination of hardware and software.

As used herein, expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

Hereinafter, various embodiments of a gene amplification chip, a gene amplification device, and a method of manufacturing a gene amplification chip will be described in detail with reference to the drawings.

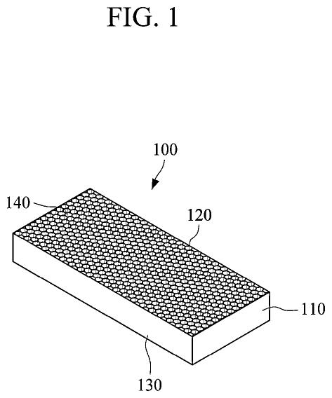

FIG. 1 is a diagram illustrating a gene amplification chip according to an example embodiment.

Referring to FIG. 1, a gene amplification chip 100 includes a substrate 110, an upper surface 120 of the substrate 110, a lower surface 130 of the substrate 110, and an array of through-holes 140.

The substrate 110 may comprise an inorganic material, such as silicon (Si), glass, polymer, metal, ceramic, and graphite, acrylic, polyethylene terephthalate (PET), polycarbonate, polystylene, and polypropylene, but is not limited thereto. The thickness of the substrate 110, that is, the length from the upper surface 120 to the lower surface 130 of the substrate 110 may be less than or equal to 1 millimeter (mm), but is not limited thereto and may vary without limitation.

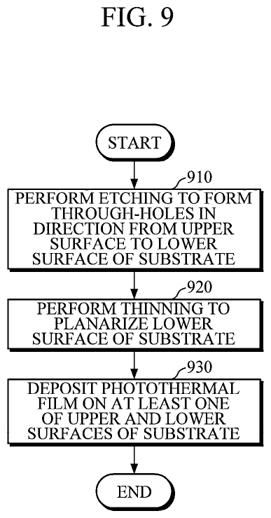

The through-holes 140 may be formed to extend from the upper surface 120 to the lower surface 130 of the substrate 110 as illustrated. Etching including deep reactive-ion etching (DRIE) and thinning including a chemical mechanical polishing (CMP) process may be performed to form the through-holes 140. A method of forming the through-holes 140 will be described in detail with reference to FIG. 9.

The volume of each through-hole 140 may be less than or equal to 1 nanoliter (nL), and the number of through-holes 140 may be at least 20,000. The through-holes 140 may be in the shape of a circular cylinder or a hexagonal cylinder, but are not limited thereto and may be formed in various shapes, such as other polygonal cylinders. When the through-holes 140 are in the shape of a hexagonal cylinder, a diagonal distance of the cross-sectional area of each through-hole 140 may be less than or equal to 100 micrometers (μm). However, characteristics, such as the number, shape, or volume of the through-holes 140, are not limited thereto, and may vary without limitation.

A gene amplification reaction occurs inside the through-holes 140. In this case, a process of reverse transcription of a ribonucleic acid (RNA) sample in each through-hole 140 using a reverse transcriptase may be performed. The gene amplification reaction may include, for example, a nucleic acid amplification reaction including at least one of a polymerase chain reaction (PCR) amplification and an isothermal amplification, an oxidation-reduction reaction, and a hydrolysis reaction. In this case, a gene may include one or two or more duplexes of RNAs, deoxyribonucleic acids (DNAs), peptide nucleic acids (PNA), or locked nucleic acids (LNAs). However, the gene is not limited thereto.

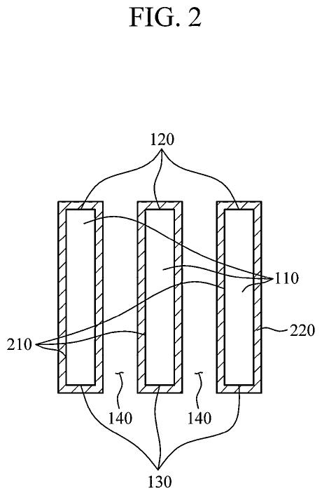

The gene amplification chip 100 may include a photothermal film 220 as shown in FIG. 2. The shape of the gene amplification chip 100 with the photothermal film 220 deposited thereon will be described with reference to FIG. 2.

FIG. 2 is a side view of a gene amplification chip with a photothermal film deposited thereon.

Referring to FIG. 2, the gene amplification chip includes a photothermal film 220 in addition to the above-described substrate 110, the upper surface 120 of the substrate 110, the lower surface 130 of the substrate 110, and the array of through-holes 140. FIG. 2 illustrates a state in which the photothermal film 220 is provided on the upper surface 120 of the substrate 110, the lower surface 130 of the substrate 110, and partition walls 210 of the through-holes 140. In this case, the photothermal film 220 may be provided in a pattern.

Alternatively, the photothermal film 220 may be deposited on only one of the upper surface 120 of the substrate 110, the lower surface 130 of the substrate 110, and the partition walls 210 of the through-holes 140, or may be deposited only on the upper surface 120 of the substrate 110 and the lower surface 130 of the substrate 110. In this case, processing complexity or manufacturing cost may be reduced as compared to when the photothermal film 220 is provided on both the upper surface 120 and lower surface 130 of the substrate 110 and the partition walls 210 of the through-holes 140.

The thickness of the photothermal film 220 may be less than or equal to 10 μm, but is not limited thereto. In addition, the photothermal film 220 may be formed as a metal layer, but is not limited thereto. The photothermal film 220 may be formed of a metal oxide material, a metalloid, or a non-metal. For example, the photothermal film 220 may be formed of a tungsten oxide-based material that has excellent infrared absorption ability and thus provides excellent photothermal conversion performance upon laser irradiation.

The photothermal film 220 may be formed by nanostructures. For example, the photothermal film 220 may be formed by nanoparticles having a diameter of less than or equal to 50 nanometers (nm) and a thickness of less than or equal to 50 nm, nanorods, nanodiscs, or nanoislands, but is limited thereto. The photothermal film 220 may be formed by various other nanostructures.

In addition, the photothermal film 220 may additionally include carbon black, visible light dyes, ultraviolet dyes, infrared dyes, fluorescent dyes, radiation-polarizing dyes, pigment, a metal compound, or other suitable absorbing materials as a photothermal conversion material.

The photothermal film 220 may receive light from, for example, a light source, and generate heat through the received light (photonic heating). In this case, the photothermal film 220 is provided on a plurality of positions of the gene amplification chip 100, so that uniform control of temperature is possible and thermal generation efficiency is increased.

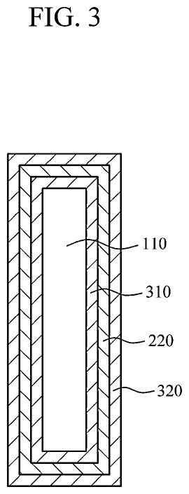

FIG. 3 is a diagram illustrating a gene amplification chip according to another example embodiment.

Referring to FIG. 3, the gene amplification chip may further include an adhesive film provided between a substrate 110 and a photothermal film 220 to improve adhesion of the photothermal film 220. There is no limit on the components of the adhesive film 310, and an adhesive may be applied to the adhesive film 310. In addition, a release paper may be attached to protect the adhesive. In addition, the adhesive film 310 may additionally include a separate configuration that improves the adhesion between the photothermal film 220 and the substrate 110.

The gene amplification chip 100 may further include an auxiliary film 320.

The auxiliary film 320 may prevent the photothermal film 220 from inhibiting a gene amplification process inside the through-holes, and may thereby protect the gene amplification process. When the photothermal film 220 is charged with electric charges, a biomaterial used in the gene amplification process may be pulled toward the photothermal film 220, which may inhibit the overall gene amplification process. The auxiliary film 320 may prevent the biomaterial from being pulled toward the photothermal film 220, and thereby protect the gene amplification process.

In addition, the auxiliary film 320 may include a material for amplifying the photothermal effect of the photothermal film 220. In this case, the auxiliary film 320 may be formed by stacking a plurality of films including various materials for amplifying the photothermal effect in a multilayer structure. The auxiliary film 320 may prevent the photothermal film 220 from inhibiting the gene amplification process inside the through-holes 140, and may amplify the photothermal effect.

The auxiliary film 320 may be provided to enclose the photothermal film 220 as illustrated, but is not limited thereto. For example, the auxiliary film 320 may not be attached to the upper surface 120 of the substrate 110 and/or the photothermal film 220 provided on the lower surface 130, but may be attached only to the photothermal film 220 provided on the partition walls of the through-holes 140. The auxiliary film 320 may be formed of any one of silicon dioxide (SiO₂), titanium dioxide (TiO₂), tantalum dioxide (TaO₂), SiN, and polymer, but is not limited thereto and may vary without limitation.

The adhesive film 310 and the auxiliary film 320 may each be provided between the through-holes 140 and the photothermal film 220 or to surround the photothermal film, using chemical vapor deposition (CVD), physical vapor deposition (PVD), atomic layer deposition method (ALD), sputtering, evaporation, etc., similar to a method in which the photothermal film 220 is provided on the upper surface 120 of the substrate 110. However, the present disclosure is not limited thereto.

Although the adhesive film 310 and the auxiliary film 320 are shown together in FIG. 3, the gene amplification chip 100 may include only one of the adhesive film 310 and the auxiliary film 320.

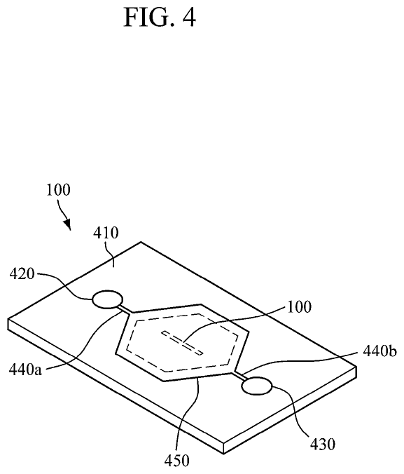

FIG. 4 is a diagram illustrating an apparatus for gene amplification according to an exemplary embodiment.

Referring to FIG. 4, an apparatus 400 for gene amplification may include a main body 410, a solution inlet 420, a solution outlet 430, a chamber 450 provided on one surface of the main body 410 and connected to the solution inlet 420 and the solution outlet 430 via fluid conduits 440a and 440b, and a gene amplification chip 100 inserted into the chamber 450. The main body 410 may include a groove through which the chamber 450 can be inserted.

A solution to be used in gene amplification reaction is loaded through the solution inlet 420. The solution may be a bio-fluid including at least one of respiratory secretions, blood, urine, sweat, tears, and saliva, a swab sample of an upper respiratory tract, or a solution obtained by dispersing such a bio-fluid or a swab sample in another medium. In this case, other media include, but are not limited to, water, saline, alcohol, phosphate buffered saline, viral transport media, and the like. In this case, the volume of the sample may be 1 microliter (μL) to 1000 μL, such as, for example, 20 μL.

The solution loaded from the solution inlet 420 may be pretreated before flowing into the chamber 450. For example, pretreatment, such as heating, chemical treatment, treatment using magnetic beads, solid phase extraction, and treatment using ultrasonic waves, may be performed. A material or structure for such pretreatment may be formed inside or outside the solution inlet 420.

In addition, the solution inlet 420 may include a field effect transistor (FET), a silicon (Si) photonics structure, a 2D micro/nano material/structure, and the like. Also, the solution inlet 420 may include a structure having optical or electrical heating characteristics for controlling the temperature of a sample. For example, the solution inlet 420 may include an optical heating material/structure that responds to a light source, such as a light emitting diode (LED), a laser, or a vertical-cavity surface-emitting laser (VCSEL), or an electric heating element, such as a Peltier element.

The apparatus 400 for gene amplification may further include a storage containing reactants for each gene to be amplified. The reactants for each gene may be lyophilized and fixed in a storage. In this case, the reactants for the gene may include, but are not limited to, reverse transcriptase, polymerase, ligase, peroxidase, primer, probe, and the like. The primer may be composed of an oligonucleotide such as, for example, a target specific single strand oligonucleotide. In addition, the probe may include an oligonucleotide such as, for example, a target-specific single-stranded oligonucleotide, a fluorescent substance, a quencher, and the like. The probe may exhibit a characteristic fluorescence signal by interacting with specific target molecules in a solution in which several different types of substances are dissolved. Such a characteristic signal may be tracked, detected, and processed for a predetermined period of time by a detector and/or a processor of the apparatus 400 for gene amplification and be used for gene detection.

Although the solution inlet 420 is shown to be circular in FIG. 4, the size, shape, and number of the solution inlets 420 may vary without limitation.

The solution loaded through the solution inlet 420 may flow into the chamber 450 along the fluid conduit 440a.

In this case, the fluid conduits 440a and 440b may each include a valve for controlling a flow of the solution. At this time, various types of microvalves for opening and closing the fluid conduits 440a and 440b may be used as the valve. For example, the microvalves may include active microvalves, such as pneumatic/thermopneumatic actuated microvalves, electrostatically actuated microvalves, piezoelectrically actuated microvalves, electromagnetically actuated microvalves, and the like, or passive microvalves that enable a system to open and close the fluid conduits depending on a direction of fluid flow or a difference in interfacial tension without any artificial external operation, and are not particularly limited.

The fluid conduit 440a may further include a filter that blocks fine particles from the sample that has been loaded to the solution inlet 420 and pretreated and passes only fluid. The filter may be a single-layer or multi-layered membrane-like filter having fine pores, and may block fine particles of a desired size according to the size of the pores. The filter may be made of a material, such as silicon, polyvinylidene fluoride (PVDF), polyethersulfone, polycarbonate, glass fiber, polypropylene, cellulose, mixed cellulose esters, polytetrafluoroethylene (PTFE), polyethylene terephthalate, polyvinyl chloride (PVC), nylon, phosphocellulose, diethylaminoethyl cellulose (DEAE), etc., but is not limited thereto. The pores may be provided in various shapes, such as a circular shape, a square shape, a slit shape, and an irregular shape caused by glass fiber.

In FIG. 4, each of the fluid conduits 440a and 440b is a straight line structure and arranged on the left and right side of the chamber 450, but the present disclosure is not limited thereto. For example, the fluid conduits 440a and 440b may have various curved shapes rather than straight lines, and may include a plurality of channels.

The solution loaded through the solution inlet 420 may be introduced into the chamber 450 along the fluid conduit 440a by the capillary action. However, the apparatus 400 for gene amplification may further include a structure for delivering a solution, such as an active/passive driving device, an electro-wetting device, or the like. In this case, the active/passive driving device may include, but is not limited to, a passive vacuum void pump, a syringe pump, a vacuum pump, a pneumatic pump, and the like.

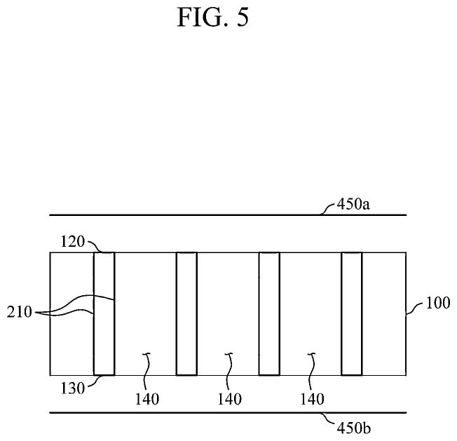

The chamber 450 may include an upper surface and a lower surface, and the gene amplification chip 100 may be inserted between the upper surface and the lower surface. Hereinafter, an example in which the gene amplification chip 100 is inserted into the chamber 450 will be described with reference to FIG. 5.

FIG. 5 is a side view of a chamber shown in FIG. 4. The gene amplification chip 100 is inserted between the upper surface 450a and the lower surface 450b. In this case, the upper surface 450a and the lower surface 450b may be glass layers, but are not limited thereto and may be composed of various components.

The upper and lower surfaces 120 and 130 of the gene amplification chip 100 and the partition walls 210 of the through-holes 140 may have the photothermal film 220 provided thereon as described in FIG. 2, or may have the adhesive film 310 and/or the auxiliary film 320 provided thereon as described in FIG. 3.

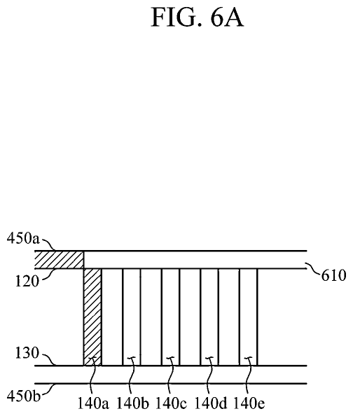

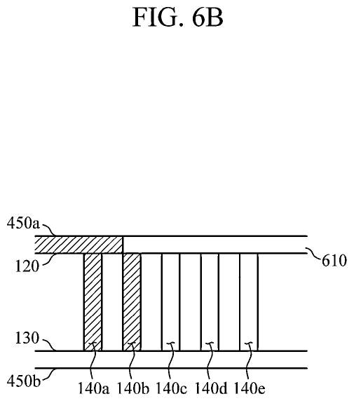

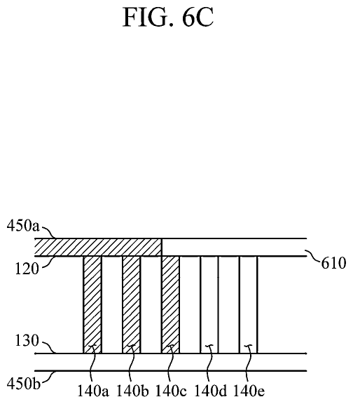

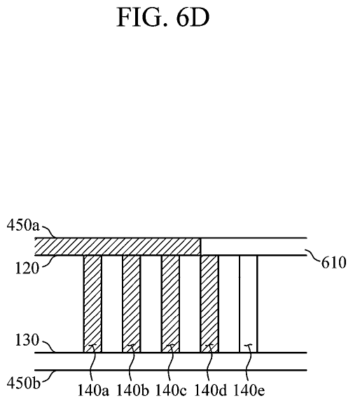

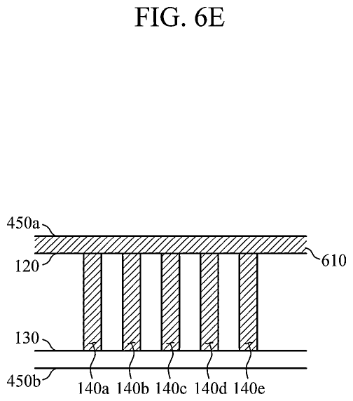

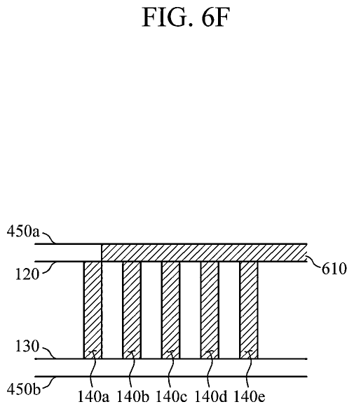

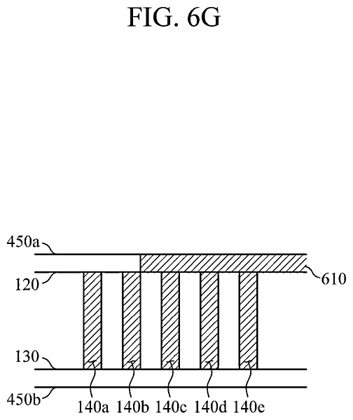

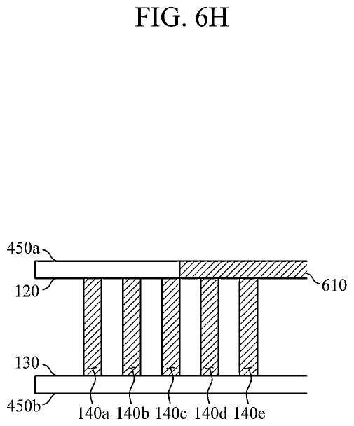

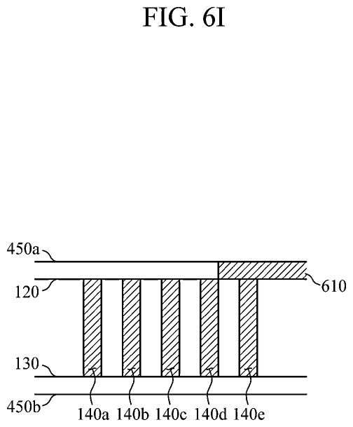

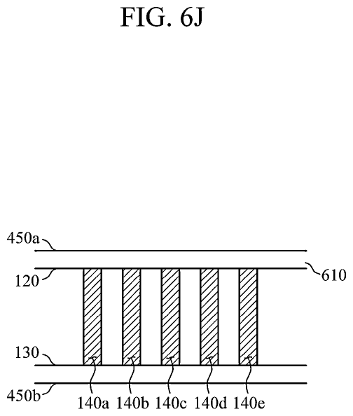

A process in which the solution is introduced into the chamber 450 will be described with reference to FIGS. 6A to 6E. FIGS. 6A to 6E illustrate a process in which a solution is injected into through-holes 140.

When the solution loaded through the solution inlet is introduced into the chamber along the fluid conduit 440a, the solution travels along a passage 610 between the upper surface 450a of the chamber 450 and the upper surface 120 of the gene amplification chip 100.

The solution introduced into the passage 610 may be injected into each through-hole 140a, 140b, 140c, 140d, and 140e by capillary action. FIG. 6A illustrates a state in which the solution introduced into the passage 610 is injected into the first through-hole 140a. Thereafter, as time passes, the solution is sequentially injected into the second through-hole 140b, the third through-hole 140c, the fourth through-hole 140d, and the fifth through-hole 140e by capillary action, and this process is illustrated in FIGS. 6B to 6E.

Alternatively, the apparatus 400 for gene amplification may include a device for performing sliding, centrifuging, stamping, or the like, so that the solution introduced into the passage 610 can be injected into each of the through-hole 140a, 140b, 140c, 140d, and 140e.

Once the solution is injected into all of the through-holes 140a, 140b, 140c, 140d, and 140e, the solution remaining in the passage 610 may be discharged to the solution outlet 430. FIGS. 6F to 6J illustrate a process in which the solution remaining in the chamber 450 other than the inside of the through-holes 140a, 140b, 140c, 140d, and 140e is discharged to the solution outlet 430.

When the solution is injected into each of the through-holes 140a, 140b, 140c, 140d, and 140e, the inside of the chamber 450 is in the state as shown in FIG. 6E. A process in which the solution remaining in the passage 610 is removed is illustrated in FIGS. 6F to 6J.

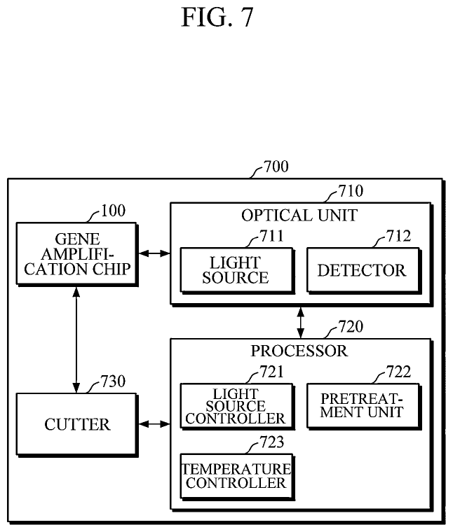

For example, the apparatus 400 for gene amplification may further include a cutter 730 as shown in FIG. 7 configured to discharge the solution remaining in the chamber 450 such as, for example, the solution remaining in the passage 610, other than the solution inside of the through-holes 140a, 140b, 140c, 140d, and 140e to the solution outlet 430 after the solution is injected into each of the through-holes 140a, 140b, 140c, 140d, and 140e. In this case, the cutter 730 may discharge the solution remaining in the passage 610 to the solution outlet 430 through the fluid conduit 440b by using oil or air. Alternatively, the solution in the passage 610 may be discharged by the capillary action of an absorption pad that may be included in the solution outlet 430.

Even when the solution remaining in the passage 610 is discharged, the solution injected into each of the through-holes 140a, 140b, 140c, 140d, and 140e does not escape to the outside of the through-holes 140a, 140b, 140c, 140d, and 140e due to the capillary action. Because the solution remaining in the passage 610 is discharged, the through-holes 140a, 140b, 140c, 140d, and 140e are no longer connected to one another by the solution so that digital PCR can be implemented and thus sensitivity and accuracy of gene amplification can be improved.

Referring back to FIG. 4, the solution remaining in the passage 610 of the chamber 450 is discharged to the solution outlet 430 along the fluid conduit 440b.

The solution outlet 430 may include an absorption pad. The absorption pad may serve to move and drain the solution using the capillary action. Including the absorption pad may facilitate controlling the moving speed of the solution. However, the present disclosure is not limited thereto, such that the flow rate and amount of the solution passing through the chamber 450 may be controlled by varying the position, size, and type of the absorption pad. For example, the reaction sensitivity may be improved by moving the sample slowly during the enzyme reaction and quickly moving the sample during washing.

FIG. 7 is a block diagram illustrating an apparatus for gene amplification according to an example embodiment.

An apparatus 700 for gene amplification may include a gene amplification chip 100, an optical unit 710, a processor 720, and a cutter 730.

The gene amplification chip 100 includes through-holes 140, and a gene amplification reaction occurs inside the through-holes 140. The gene amplification chip 100 is described in detail above, and thus the description thereof will be omitted.

The optical unit 710 measures an optical signal while a gene amplification reaction occurs inside each through-hole 140 of the gene amplification chip 100. In this case, the optical signal includes a fluorescent signal, a phosphorescent signal, an extinction signal, a surface plasmon resonance signal, and the like. The optical unit 710 may include a light source 711 and a detector 712.

The light source 711 may emit light to a photothermal film 220 of the gene amplification chip 100. The light source may include an LED, a laser, a VCSEL, and the like, but is not limited thereto. In addition, the light emitted by the light source 711 may include wavelengths in various regions. For example, the light source 711 may emit light having a wavelength in the ultraviolet (UV) to infrared (IR) range, but is not limited thereto.

The detector 712 may detect an optical signal emitted from an amplified target gene. The detector 712 may include a photomultiplier tube, a photo detector, a photomultiplier tube array, a photo detector array, and a complementary metal-oxide semiconductor (CMOS) image sensor, and the like, but is not limited thereto.

The detector 712 may use the fluorescence reflection of the photothermal film 220 when detecting fluorescence emitted from the amplified gene. For example, when a photothermal film 220 made of a constituent material with high reflectivity is deposited on the partition walls 210 of the through-holes 140 of the gene amplification chip 100, the photothermal film 220 may reflect fluorescence emitted from the amplified gene inside the through-hole 140 in the direction of the detector 712. At this time, the detector 712 may detect fluorescence reflected from the photothermal film 220.

In addition, the optical unit 710 may further include a filter for passing a specific wavelength, a mirror for adjusting fluorescence emitted from the target gene to be directed toward the detector, a lens for condensing fluorescence emitted from the target gene, and the like.

While the gene amplification reaction is performed in each through-hole 140 of the gene amplification chip 100, an optical signal may be measured by the light source 711, the detector 712, and/or the processor 720 of the apparatus 700 for gene amplification, and the amplified gene may be detected based on the measured optical signal. In this case, the optical signal includes a fluorescent signal, a phosphorescent signal, an extinction signal, a surface plasmon resonance signal, and the like. The apparatus 700 for gene amplification may be used to detect the presence or absence of a target DNA template, quantitative information, and the like, during the replication process of polymerase.

The processor 720 may be electrically connected to the optical unit 710, and may receive the optical signal from the detector 712 and analyze the received optical signal. For example, the processor 720 may quantify the gene by analyzing a digital nucleic acid amplification result detected by the detector 712 based on the Poisson distribution.

The processor 720 may include a light source controller 721.

The light source controller 721 may control whether to drive the light source 711 and the driving condition of the light source 711. The light source controller 721 may heat and cool the photothermal film 220 by driving the light source in an on-off manner. As the photothermal film 220 is heated and cooled, thermal cycling occurs and the target gene may thus be amplified. In addition, the light source controller 721 may control at least one of the type, wavelength, current intensity, duration, and on-off interval of light of the light source 711.

The processor 720 may further include a pretreatment unit 722 and/or a temperature controller 723.

The pretreatment unit 722 may perform pretreatment on the sample loaded in the solution inlet, such as heating, chemical treatment, treatment using magnet beads, solid phase extraction, treatment using ultrasonic waves, and the like. To this end, the pretreatment unit 722 may include various materials or structures for pretreatment, such as magnetic beads, an ultrasonic device, an optical/electric heating device, and the like which are disposed inside and/or outside of the solution inlet 420, and may control the materials or structures. At least some functions of the pretreatment unit 722 may be integrated into the processor 720.

The temperature controller 723 may adjust the temperature of the solution in the solution inlet 420 or in the fluid conduit 440a as shown in FIG. 4. For example, when the solution is loaded in the solution inlet 420, the temperature controller 723 may control the temperature of the sample to maintain an isothermal temperature equal to or greater than 95° C. In addition, when the solution moves along the fluid conduit, the temperature controller 723 may control the temperature of the solution to remain within a predetermined range.

The temperature controller 723 may include a material or structure for adjusting the temperature which may be provided inside or outside the solution inlet 420 or the fluid conduit of the apparatus 700 for gene amplification. For example, an electric heating unit for electrically heating the solution may be formed inside the solution inlet 420 or the fluid conduit 440a. The electric heating unit may include, for example, a heating element and/or a Peltier element. Also, the temperature controller 723 may include a temperature sensor disposed inside or outside the apparatus 700 for gene amplification to measure the temperature of the solution present in the solution inlet 420 or in the fluid conduit 440a. In this case, the temperature sensor may include a thermocouple having a bimetallic junction that generates a temperature-dependent electric motor force (EMF), a resistive thermometer including a material having an electrical resistance proportional to temperature, thermistors, IC temperature sensor, IR temperature sensor, IR cameras, quartz thermometers, and the like.

As described above with reference to FIGS. 6F to 6J, after the solution is injected into each through-hole 140, the cutter 730 may discharge the solution remaining in the chamber 450 other than the inside of the through-holes to the solution discharge. In this case, a portion of the space within the chamber 450 excluding the inside of the through-holes 140 may be the passage 610 of FIGS. 6A to 6J.

The cutter 730 may discharge the solution remaining in the passage 610 to the solution outlet 430 through the fluid conduit 440a by using oil or air. Since the solution remaining in the passage (610 is discharged, the through-holes 140 are no longer connected to one another by the solution so that digital PCR can be implemented and thus sensitivity and accuracy of gene amplification can be improved.

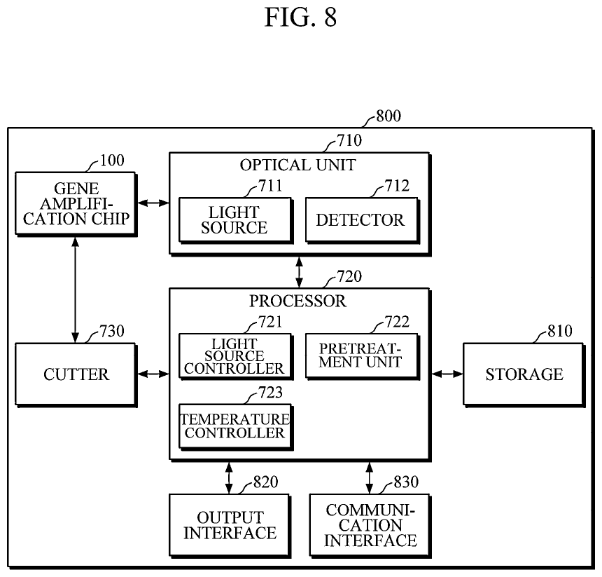

FIG. 8 is a block diagram illustrating an apparatus for gene amplification according to another example embodiment. Referring to FIG. 8, an apparatus 800 for gene amplification according to an example embodiment may further include a storage 810, an output interface 820, and a communication interface 830 in addition to the components of the apparatus 700 for gene amplification in accordance with the example embodiment of FIG. 7.

The storage 810 may output, for example, a variety of reference information for gene amplification and/or the gene amplification results. The storage 810 may include at least one type of storage medium, such as a flash memory type, a hard disk type, a multimedia card micro type, a card type memory (e.g., secure digital (SD) or extreme digital (XD) memory), random access memory (RAM), static random access memory (SRAM), read-only memory (ROM), electrically erasable programmable read-only memory (EEPROM), programmable read-only memory (PROM), a magnetic memory, a magnetic disk, and an optical disk.

The output interface 820 may output, for example, a gene amplification process, a gene amplification, an analysis result. The output interface 820 may provide information to a user using visual, auditory, and tactile methods, such as a visual output module (e.g., a display), an audio output module (e.g., a speaker), a haptic module, and the like.

The communication interface 830 may communicate with an external device. For example, the communication interface 830 may transmit data generated in the apparatus 700 or 800 for gene amplification, for example, a gene detection result, to the external device, and may receive data for gene detection from the external device. Here, the external device may be medical equipment, a printer to print out results, or a display to display the results. In addition, the external device may be a digital TV, a desktop computer, a cellular phone, a smartphone, a tablet PC, a laptop computer, a personal digital assistant (PDA), a portable multimedia player (PMP), a navigation system, an MP3 player, a digital camera, a wearable device, and the like, but is not limited thereto.

The communication interface 830 may communicate with the external device by using various communication techniques such as Bluetooth communication, Bluetooth Low Energy (BLE) communication, Near Field Communication (NFC), WLAN communication, Zigbee communication, Infrared Data Association (IrDA) communication, wireless fidelity (Wi-Fi) Direct (WFD) communication, Ultra-Wideband (UWB) communication, Ant+ communication, Wi-Fi communication, Radio Frequency Identification (RFID) communication, 3G communication, 4G communication, 5G communication, and the like. However, these are merely examples, and the present disclosure is not limited thereto.

FIG. 9 is a flowchart illustrating a method of manufacturing the gene amplification chip 100 of FIG. 1 according to an example embodiment. A process in which through-holes 140 are formed on the substrate 110 and the photothermal film 220 is provided will be described with reference to FIG. 9.

First, the substrate may be etched to form through-holes 140 in the direction from the upper surface 120 to the lower surface 130 of the substrate 110 in operation 910. Etching may be performed starting from the upper surface 120 toward the lower surface 130 so as to form the through-holes 140. As a specific method of etching, deep reactive-ion etching (DRIE) or reactive-ion etching (RIE) may be used. However, the method is not limited thereto, and the type and method of etching may vary. For example, wet etching, dry etching, and gas etching may be used.

Then, thinning may be performed to planarize the lower surface 130 of the substrate 110 in operation 920. At this time, the thinning may include a CMP process, and the flatness, uniformity, and polishing rate in the CMP process may be specified without limitation. However, the thinning is not limited to the CMP process, and grinding and other polishing may be used.

Then, when the through-holes are formed through operations 910 and 920, a photothermal film 220 may be deposited on at least one of the upper surface 120 and the lower surface 130 of the substrate 110 in operation 930. In this case, an operation of further depositing the photothermal film 220 on partition walls of the through-holes 140 may be included. The photothermal film 220 may be deposited in a pattern.

Specific deposition methods of the photothermal film 220 include CVD, PVD, ALD, sputtering, evaporation, and the like, but is not limited thereto.

The example embodiments can be implemented by computer-readable code that is stored in a non-transitory computer-readable medium and executed by a processor. Code and code segments constituting a computer program can be easily inferred by a computer programmer skilled in the art. The computer-readable medium includes all types of record media in which computer readable data are stored. Examples of the computer-readable medium include a ROM, a RAM, a CD-ROM, a magnetic tape, a floppy disk, and an optical data storage. Further, the computer-readable medium may be implemented in the form of a carrier wave such as Internet transmission. In addition, the computer-readable medium may be distributed to computer systems over a network, in which computer readable code may be stored and executed in a distributed manner.

Although various example embodiments have been described, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A gene amplification chip comprising:
a substrate;
a through-hole array including through-holes that extend from an upper surface of the substrate to a lower surface of the substrate and in which a gene amplification reaction occurs; and
a photothermal film provided on at least one of the upper surface and the lower surface of the substrate, the photothermal film being configured to generate heat using light.

2. The gene amplification chip of claim 1, wherein the substrate comprises silicon (Si), glass, polymer, or metal.

3. The gene amplification chip of claim 1, wherein a thickness of the substrate is less than or equal to 1 millimeter (mm).

4. The gene amplification chip of claim 1, wherein each of the through-holes has a volume of less than or equal to 1 nanoliter (nL).

5. The gene amplification chip of claim 1, wherein a number of the through-holes is equal to or greater than 20,000.

6. The gene amplification chip of claim 1, wherein the through-holes have a circular cylinder shape or a polygonal cylinder shape.

7. The gene amplification chip of claim 1, wherein each of the through-holes has a hexagonal cylinder shape, and diagonal distance of a cross-sectional area of each of the through-holes is less than or equal to 100 micrometers (μm).

8. The gene amplification chip of claim 1, wherein the photothermal film has a thickness that is less than or equal to 10 micrometers (μm).

9. The gene amplification chip of claim 1, wherein the photothermal film is provided on partition walls of each of the through-holes.

10. The gene amplification chip of claim 1, wherein the photothermal film comprises a metal layer.

11. The gene amplification chip of claim 1, wherein the photothermal film comprises at least one of nanoparticles, nanorods, nanodisks, and nanoislands.

12. The gene amplification chip of claim 1, further comprising an auxiliary film attached to the photothermal film.

13. The gene amplification chip of claim 12, wherein the auxiliary film is comprises silicon dioxide (SiO₂), titanium dioxide (TiO₂), tantalum dioxide (TaO₂), silicon nitride (SiN), or polymer.

14. The gene amplification chip of claim 1, further comprising an adhesive film disposed between the substrate and the photothermal film to provide adhesion of the photothermal film.

15. An apparatus for gene amplification, the apparatus comprising:
a main body;
a gene amplification chip;
a chamber provided on a side of the main body and connected to a solution inlet and a solution outlet through fluid conduits, the chamber being configured to allow the gene amplification chip to be inserted therein;
a light source configured to emit light to the gene amplification chip; and
a detector configured to detect fluorescence emitted from an amplified gene,
wherein the gene amplification chip comprises:
a substrate;
a through-hole array including through-holes that extend from an upper surface of the substrate to a lower surface of the substrate and in which a gene amplification reaction occurs; and
a photothermal film provided on at least one of the upper surface and the lower surface of the substrate, the photothermal film being configured to generate heat using light.

16. The apparatus of claim 15, wherein the chamber comprises an upper surface and a lower surface, and the gene amplification chip is inserted between the upper surface and the lower surface.

17. The apparatus of claim 15, wherein when a solution is loaded through the solution inlet and introduced into the chamber along the fluid conduits, the solution is injected into the through-holes by capillary action.

18. The apparatus of claim 15, further comprising a cutter configured to discharge solution remaining in the chamber other than in the inside of the through-holes to the solution outlet after the solution loaded through the solution inlet is injected into the through-holes.

19. The apparatus of claim 15, further comprising a light source controller configured to heat and cool the photothermal film by driving the light source in an on-off manner.

20. The apparatus of claim 15, wherein the photothermal film reflects the fluorescence emitted from the gene amplified inside the through-holes in a direction of the detector.

21. A method of manufacturing a gene amplification chip, the method comprising:
forming through-holes in a substrate, the through-holes extending in a direction from an upper surface to a lower surface of the substrate;
planarizing the lower surface of the substrate using a chemical mechanical polishing (CMP) process; and
depositing a photothermal film on at least one of the upper surface and the lower surface of the substrate.

22. The method of claim 21, further comprising depositing the photothermal film on partition walls of each of the through-holes.
